SEQUENCE LISTING (6 sequences)

SEQ ID NO: 1 - DNA, 40 bp, Artificial Sequence
  Aptamer A
ggtgagttaaggaatcagcggctcagacgacccgacgcac

SEQ ID NO: 2 - DNA, 40 bp, Artificial Sequence
  Aptamer B
aaacaaccctaatgggagtataatggtattcagtttggta

SEQ ID NO: 3 - DNA, 40 bp, Artificial Sequence
  Aptamer C
gtagctgtccactaatttatatacgatgagaaccatgcct

SEQ ID NO: 4 - DNA, 40 bp, Artificial Sequence
  Aptamer D
catgccgctgagcttaggcaatttccatatttcaggtaga

SEQ ID NO: 5 - DNA, 23 bp, Artificial Sequence
  primer
tgaggtaccagctgctgctgcat

SEQ ID NO: 6 - DNA, 20 bp, Artificial Sequence
  primer
gacctcgtcggtctagacgc

(19) United States
(12) Patent Application Publication
(10) Pub. No.: US 2023/0028090 A1
(43) Pub. Date: Jan. 26, 2023

(54) Method Of Using Aptamer For Detecting Glycated Hemoglobin In Whole Blood And Nanoelectronic Aptasensor

(71) Applicant: National Taiwan University, Taipei (TW)

(72) Inventor: Yit-Tsong Chen, Taipei City (TW)

(73) Assignee: National Taiwan University, Taipei (TW)

(21) Appl. No.: 17/381,622

(22) Filed: Jul. 21, 2021

(51) Int. Cl. G01N 33/72; C12N 15/115; G01N 33/543; G01N 33/553

(52) U.S. Cl. CPC G01N 33/723; C12N 15/115; G01N 33/54346; G01N 33/553; B82Y 35/00

(57) ABSTRACT

Provided is a method of using an aptamer for detecting a glycated hemoglobin in a whole blood, the method includes that the aptamer is provided, the aptamer includes a DNA sequence selected from the group consisting of derived sequences of SEQ ID NOs: 1, 2, 3, and 4, in which the derived sequences refer to that 3′ end and/or 5′ end of the derived sequences are modified, and the derived sequences have 90% identity to the SEQ ID NOs: 1, 2, 3, and 4. The aptamer and the whole blood are contacted. A concentration of a conjugate of the aptamer and the glycated hemoglobin is estimated. Provided also is a nanoelectronic aptasensor including the above aptamer.

### CROSS-REFERENCE TO RELATED APPLICATION

The Sequence Listing associated with this application is filed in electronic format via EFS-Web and is hereby incorporated by reference into the specification in its entirety. The name of the text file containing the Sequence Listing is NP-29356-US_SEQ.txt. The size of the text file is 1.31 KB, and the text file was created on Jul. 19, 2021.

### BACKGROUND

### Field of Invention

The present invention relates to a method of using aptamer for detecting glycated hemoglobin in whole blood and a nanoelectronic aptasensor.

### Description of Related Art

Diabetes mellitus is one of the most common metabolic disorders in the world and has a major impact not only on patients' quality of life but also on the efficacy of medical care. The routine management for diabetes mellitus requires monitoring the fasting blood glucose concentration in the range of around 4 to 8 mM using a portable glucose meter. However, variations in blood glucose levels are closely linked to diet, exercise, and blood insulin levels. On the other hand, the glycated hemoglobin A1c (HbA1c) represents the average blood glucose level in the recent three months, which is a crucial indicator for long-term sugar control, free from diurnal glucose fluctuations, and unaffected by recent exercise or stress levels.

Various analytical techniques, such as ion-exchange chromatography, boronate affinity chromatography, electrophoresis, and immunoassay, have been used to quantify the HbA1 c level. However, most of these techniques are feasible only in specialized laboratories, leading to several disadvantages, including poor efficiency, costly instruments, and cumbersome operational procedures. Therefore, the disadvantage of the prior art should be resolved.

### SUMMARY

The present disclosure provides a method of using an aptamer for detecting a glycated hemoglobin in a whole blood, the method comprises that the aptamer is provided, the aptamer comprises a DNA sequence selected from the group consisting of derived sequences of SEQ ID NOs: 1, 2, 3, and 4, in which the derived sequences refer to that 3′ end and/or 5′ end of the derived sequences are modified, and the derived sequences have 90% identity to the SEQ ID NOs: 1, 2, 3, and 4. The aptamer and the whole blood are contacted. A concentration of a conjugate of the aptamer and the glycated hemoglobin are estimated.

In some embodiments, the whole blood is from a human being.

In some embodiments, the glycated hemoglobin comprises a glycated peptide.

In some embodiments, the glycated peptide is D-fructose-valine-histidine-leucine-threonine-proline-glutamic acid.

In some embodiments, the D-fructose comprises β-D-fructopyranose, β-D-fructofuranose, α-D-fructofuranose, or α-D-fructopyranose.

The present disclosure also provides a nanoelectronic aptasensor, which comprises a substrate, a transistor, a plurality of silicon nanowires, and a plurality of aptamers. The transistor is disposed on the substrate, the transistor comprising a source electrode, a drain electrode, and a gate electrode. The plurality of silicon nanowires are disposed on the substrate, two ends of the plurality of silicon nanowires respectively connect to the source electrode and the drain electrode, and each one of the plurality of silicon nanowires is separated to each other, in which each one of the plurality of silicon nanowires is single-crystalline. The plurality of aptamers are disposed on the plurality of silicon nanowires, and the aptamer comprises a DNA sequence selected from the group consisting of derived sequences of SEQ ID NOs: 1, 2, 3, and 4, in which the derived sequences refer to that 3′ end and/or 5′ end of the derived sequences are modified, and the derived sequences have 90% identity to the SEQ ID NOs: 1, 2, 3, and 4. A leakage current of the nanoelectronic aptasensor is from 10 pA to 100 pA measured when the aptasensor is covered by one fold phosphate buffered saline (1×PBS, containing 137 mM NaCl, 2.7 mM KCl, 10 mM Na₂HPO₄, and 2 mM KH₂PO₄, pH 7.4) buffer solution at a solution gate-source voltage of 1 V, and a dissociation constant (Kd) without co-modification of polyethylene glycol (PEG) is from 39 nM to 53 nM.

In some embodiments, the aptasensor further comprises a silane-based self-assembled monolayer (SAM) disposed on the plurality of silicon nanowires, and the silane-based SAM comprises 3-mercaptopropyl trimethoxysilane (MPTMS), propyltrimethoxysilane (PTMS), or a combination thereof. The plurality of aptamers are conjugated with the MPTMS, and a ratio of the plurality of aptamers and PTMS is 1:4.

In some embodiments, the plurality of aptamers are conjugated with the MPTMS by disulfide bonds.

In some embodiments, the silane-based SAM further comprises PEG, and a ratio of the plurality of aptamers, PEG and PTMS is selected from the group consisting of 2:1:3, 1:1:3, 1:2:3, 1:4:3, and 1:6:3.

In some embodiments, the plurality of aptamers are conjugated with the MPTMS by disulfide bonds.

In some embodiments, each one of the plurality of silicon nanowires has a diameter in a range of 20 nm to 30 nm.

In some embodiments, the plurality of silicon nanowires is p-type semiconductor or n-type semiconductor.

In some embodiments, the plurality of silicon nanowires are manufactured by the following steps comprising: providing a Si wafer chip incubated in poly-L-lysine solution for 10 minutes; incubating gold nanoparticles onto the Si wafer chip for 10 seconds; cleaning the Si wafer chip by oxygen plasma 100 W and 50 sccm for 300 seconds; and growing the plurality of silicon nanowires by the chemical-vapor-deposition reaction in 10 sccm Ar, 6 sccm SiH₄, and 15 sccm B₂H₆.

In some embodiments, the nanoelectronic aptasensor is manufactured by the following steps comprising: depositing the plurality of silicon nanowires on an oxide layer of a Si wafer; coating photoresist layers on the deposited Si wafer; developing the coated Si wafer by a developer for 1 minute; etching a portion of silica sheathes on the plurality of silicon nanowires by a buffer of oxide etching for 5 seconds; depositing the source and drain electrodes on the plurality of silicon nanowires; and annealing in forming gas for 3 minutes.

In some embodiments, the plurality of aptamers disposed on the plurality of silicon nanowires are manufactured by the following step comprising: incubating the plurality of aptamers with the plurality of silicon nanowires for 1 hour.

In some embodiments, a detection range of a glycated hemoglobin in whole blood by the aptamers of SEQ ID NO: 1 is from 10⁻⁹ to 1.2×10⁻⁶ M.

In some embodiments, a material of the source electrode and the drain electrode is selected from the group consisting of nickel and aluminum.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion. The invention can be more fully understood by reading the following detailed description of the embodiment, with reference made to the accompanying drawings as follows.

FIG. 1 is a schematic illustration of a systematic evolution of ligands by exponential enrichment (SELEX) cycle for the selection of glycated peptide (GP)-specific DNA aptamers according to some embodiments of the present disclosure.

FIG. 2 is a schematic view of secondary structures of four potential GP-specific aptamers, i.e., Aptamer A (also denoted by Apt_{GP}), Aptamer B, Aptamer C, and Aptamer D according to some embodiments of the present disclosure.

FIG. 3 is a schematic view of secondary structure of Apt_{GP} according to some embodiments of the present disclosure.

FIG. 4 is a schematic view of a chemical structure of the GP ligand is illustrated according to some embodiments of the present disclosure.

FIG. 5 is a top view of a single chip contains six multiple-parallel-connected silicon nanowire field-effect transistors (MPC SiNW-FET) devices according to some embodiments of the present disclosure, and scale bar refers to 100 μm.

FIG. 6 is a close-up view of FIG. 5 according to some embodiments of the present disclosure.

FIG. 7 is a cross-sectional view of the MPC SiNW-FET device along line AA of FIG. 6 according to some embodiments of the present disclosure.

FIG. 8 is a schematic view of an experimental set-up for detecting GP or HbA1c according to some embodiments of the present disclosure.

FIG. 9 is an output curve of the MPC SiNW-FET showing an ohmic contact according to some embodiments of the present disclosure.

FIG. 10 is a transfer curve of the MPC SiNW-FET according to some embodiments of the present disclosure.

FIG. 11 is a transfer curve acquired for MPTMS/SiNW-FET (before modifying Apt_{GP}) and Apt_{GP}/SiNW-FET (after modifying Apt_{GP}) by scanning a back gate voltage according to some embodiments of the present disclosure.

FIG. 12 is shifts of the transfer curve of an Apt_{GP}/SiNW-FET measured in response to various concentrations of GP in 0.1×PBS according to some embodiments of the present disclosure.

FIG. 13 is a normalized ΔV_{g}^{cal}/ΔV_{g}^{cal,max} (ΔV_{g}^{cal} representing the change in gate voltage (V_{g}) relative to the buffer solution and termed the calibrated response, and ΔV_{g}^{cal,max} representing the saturated ΔV_{g}^{cal}) vs. concentrations of GP (C_{GP}) plot obtained by taking the data points from FIG. 12 at V_{g}=0 V according to some embodiments of the present disclosure.

FIG. 14 is shifts of the transfer curve of a PEG:Apt_{GP}/SiNW-FET measured in response to HbA1c at 10⁻⁹ to 1.2×10⁻⁶ M in 1×PBS according to some embodiments of the present disclosure.

FIG. 15 is a plot of normalized ΔV_{g}^{cal}/ΔV_{g}^{cal,max} vs. C_{HbA1c} by taking the data points from FIG. 14 at V_{g}=0 mV according to some embodiments of the present disclosure.

FIG. 16 is shifts of the transfer curve of a PEG:Apt_{GP}/SiNW-FET measured in response to HbA1c level in the hemolytic blood specimen (upper panel) or serum (lower panel) of Subject 1 according to some embodiments of the present disclosure.

FIG. 17 is a plot of electrical responses (ΔV_{g}^{cal}/ΔV_{g}^{cal,max}) as a function of the diluted C_{HbA1c} in the blood specimen or serum collected from FIG. 16 at V_{g}=0 mV according to some embodiments of the present disclosure.

FIG. 18 is a table of the C_{HbA1c} values in human blood samples of Subjects 1 to 10 measured by PEG:Apt_{GP}/SiNW-FET and compared with those determined by conventional capillary electrophoresis according to some embodiments of the present disclosure.

### DETAILED DESCRIPTION

The following disclosure provides detailed description of many different embodiments, or examples, for implementing different features of the provided subject matter. These are, of course, merely examples and are not intended to limit the invention but to illustrate it. In addition, various embodiments disclosed below may combine or substitute one embodiment with another, and may have additional embodiments in addition to those described below in a beneficial way without further description or explanation. In the following description, many specific details are set forth to provide a more thorough understanding of the present disclosure. It will be apparent, however, to those skilled in the art, that the present disclosure may be practiced without these specific details.

Further, spatially relative terms, such as “beneath,” “over” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, or “includes” and/or “including” or “has” and/or “having” when used in this specification, specify the presence of stated features, regions, integers, steps, operations, elements, and/or components, but do not preclude the presence or addition of one or more other features, regions, integers, steps, operations, elements, components, and/or groups thereof.

In Vitro Selection of DNA Aptamers by SELEX

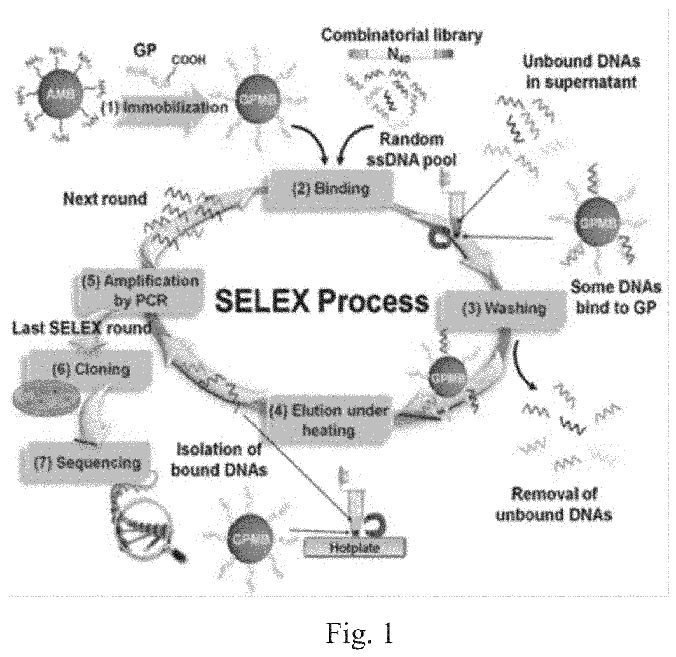

In some embodiments, to select an HbA1c-specific aptamer, we employed the SELEX method to isolate a DNA aptamer, which holds a specific affinity toward the glycated peptide at the N-terminal of HbA1c with the amino acid sequence of Fru-Val-His-Leu-Thr-Pro-Glu-COOH (referred to as GP hereafter). As illustrated schematically in FIG. 1, the amine-tagged magnetic beads (AMB) were first modified with GP. A library of ssDNAs with random sequences involving a central variable region flanked by two primer-binding regions (i.e., 5′-TGA GGT ACC AGC TGC TGC TGC AT—N40—GAC CTC GTC GGT CTA GAC GC-3′, refers to SEQ ID NO: 5—N40—SEQ ID NO: 6) was mixed with the GP-immobilized magnetic beads (GPMB). After incubation, the unbound ssDNAs in the supernatant were removed by washing the ssDNAs-bounded GPMB with 1×PBS. Upon heating at 95° C. for 10 min, the bound ssDNAs, eluted from the surface of GPMB, were amplified via polymerase chain reaction (PCR) and purified through electrophoresis. Subsequently, these ssDNAs served as a starting library for the next round of the SELEX cycle. With five rounds in all, the final ssDNAs that remained after the last SELEX cycle were identified by cloning and sequencing to exist 217 different DNA sequences.

Oligreen Assay for SELEX-isolated DNA Aptamers

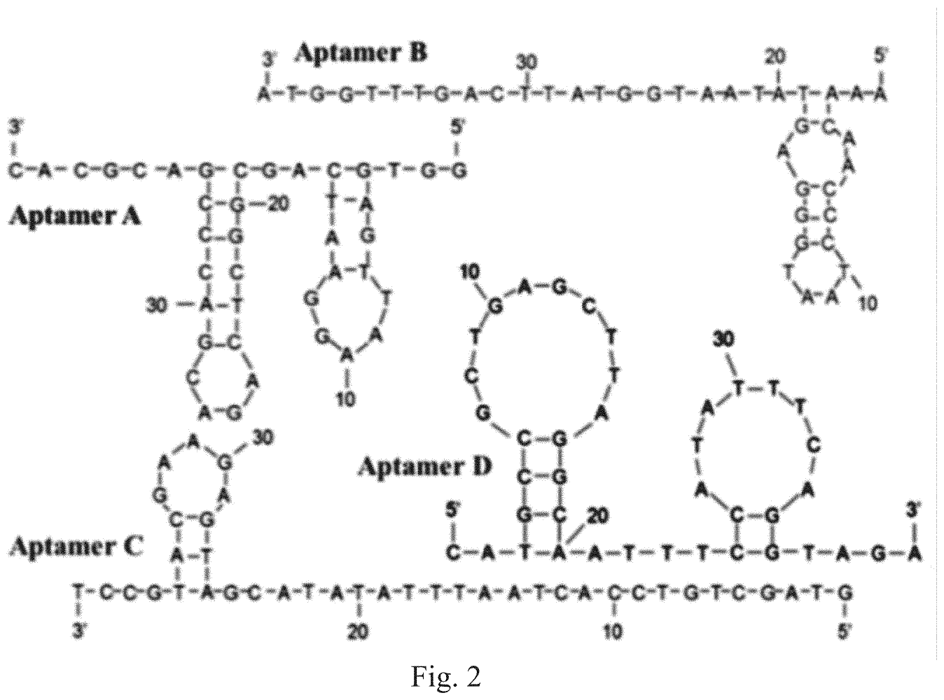

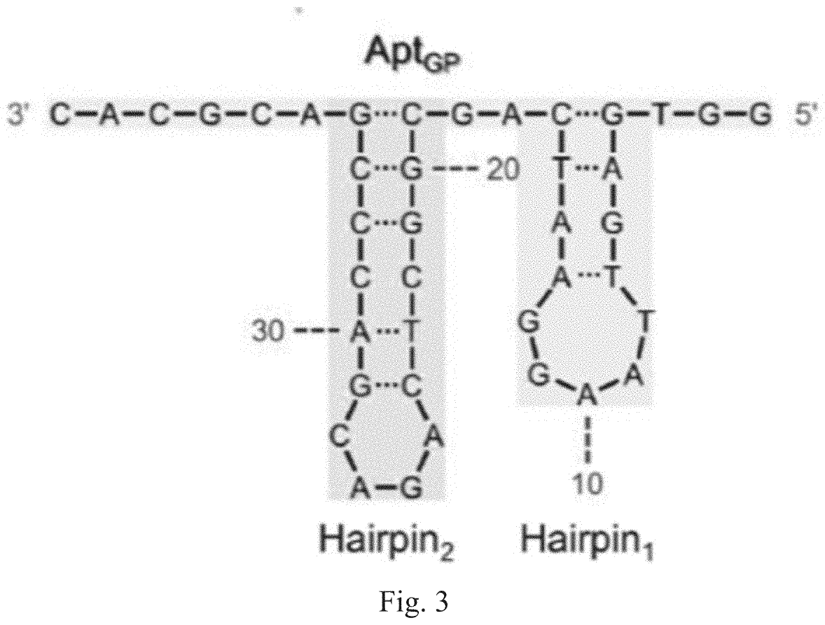

In some embodiments, after sequencing the 217 SELEX-isolated DNA aptamers, the comparative binding affinities of these aptamers to GP were determined by a fluorescent assay using OliGreen as a fluorescent reporter. Based on the OliGreen test, four DNA aptamers of different sequences with unique secondary structures (Aptamer A SEQ ID NO: 1, Aptamer B SEQ ID NO: 2, Aptamer C SEQ ID NO: 3, Aptamer D SEQ ID NO: 4; FIG. 2) exhibited the most prominent binding affinities to GP among the total 217 sequences. Among these four aptamers, Aptamer A with the secondary structure shown in FIG. 3 has excellent target specificity and the highest binding affinity towards 1 μM GP against 10 μM NGP (i.e., a non-glycated peptide of NH₂-Val-His-Leu-Thr-Pro-Glu-COOH) as compared with the other three competitors of Aptamers B, C, and D. Accordingly, Aptamer A was selected as the receptor modified on a SiNW-FET device (referred to as Apt_{GP}/SiNW-FET) for specifically recognizing GP, or the whole HbA1c protein, in the later biosensing experiments.

In some embodiments, the aptamers are selected from a derived sequence of SEQ ID NO. 1, a derived sequence of SEQ ID NO. 2 a derived sequence of SEQ ID NO. 3, and a derived sequence of SEQ ID NO. 4. The term “derived sequence” refers to that 3′ end and/or 5′ end of the oligonucleotide sequence in the present disclosure is modified, and part or all of the oligonucleotide sequence can be reserved. In other words, the derived sequence of SEQ ID NO. 1 refers to that when the sequence length of the SEQ ID NO. 1 can be increased or decreased, the oligonucleotide sequence having about 1% to 10% variation can be tolerated, i.e., 2%, 3%, 4%, 5%, 6%, 7%, 8%, or 9%. The derived sequence of SEQ ID NO. 2 refers to that when the sequence length of the SEQ ID NO. 2 can be increased or decreased, the oligonucleotide sequence having about 1% to 10% variation can be tolerated, i.e., 2%, 3%, 4%, 5%, 6%, 7%, 8%, or 9%. The derived sequence of SEQ ID NO. 3 refers to that when the sequence length of the SEQ ID NO. 3 can be increased or decreased, the oligonucleotide sequence having about 1% to 10% variation can be tolerated, i.e., 2%, 3%, 4%, 5%, 6%, 7%, 8%, or 9%. The derived sequence of SEQ ID NO. 4 refers to that when the sequence length of the SEQ ID NO. 4 can be increased or decreased, the oligonucleotide sequence having about 1% to 10% variation can be tolerated, i.e., 2%, 3%, 4%, 5%, 6%, 7%, 8%, or 9%.

Molecular Docking

In some embodiments, understanding the high-affinity interaction between Apt_{GP} and GP is crucial for using this newly synthesized Apt_{GP} as a sensitive molecular probe for HbA1c-specific biosensors. The Apt_{GP} is composed of an intra-helical structure of hairpin1, hairpin2, and loop (FIG. 3).

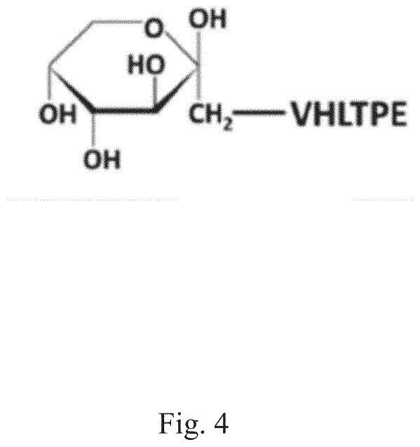

The structures of the D-fructose tautomers at equilibrium are β-D-fructopyranose, β-D-fructofuranose, α-D-fructofuranose, and only a trace of α-D-fructopyranose, which are sensitive to the environmental polarity. In some examples, the VHLTPE peptide was glycated with four D-fructose tautomers, separately (FIG. 4 only shows VHLTPE peptide with β-D-fructopyranose, others are not shown).

Synthesis of Single-Crystalline SiNWs

In some embodiments, single-crystalline boron-doped SiNWs (Si:B=4000:1) were synthesized in chemical-vapor-deposition (CVD) reaction with gold nanoparticles (Au NPs) of 20 nm in diameter as a catalyst via the vapor-liquid-solid (VLS) growth mechanism. Au NPs were dispersed evenly onto an Si wafer chip, which had been incubated in poly-L-lysine solution (0.1%) for 10 min to increase the adhesion of Au NPs. After the deposition of Au NPs, the substrate was washed with deionized water, blown dry with N₂ gas, and cleaned in oxygen plasma (100 W and 50 sccm O₂ for 300 s). The p-type SiNWs were grown in the CVD reaction at 460° C. for 12.5 min in 10 sccm Ar, 6 sccm SiH₄ (10% in He), and 15 sccm B₂H₆ (100 ppm in He) at a total chamber pressure of 25 torr. The diameters of the as-synthesized SiNWs were generally between 20 and 30 nm.

Device Fabrication and Electrical Characterizations of SiNW-FETs

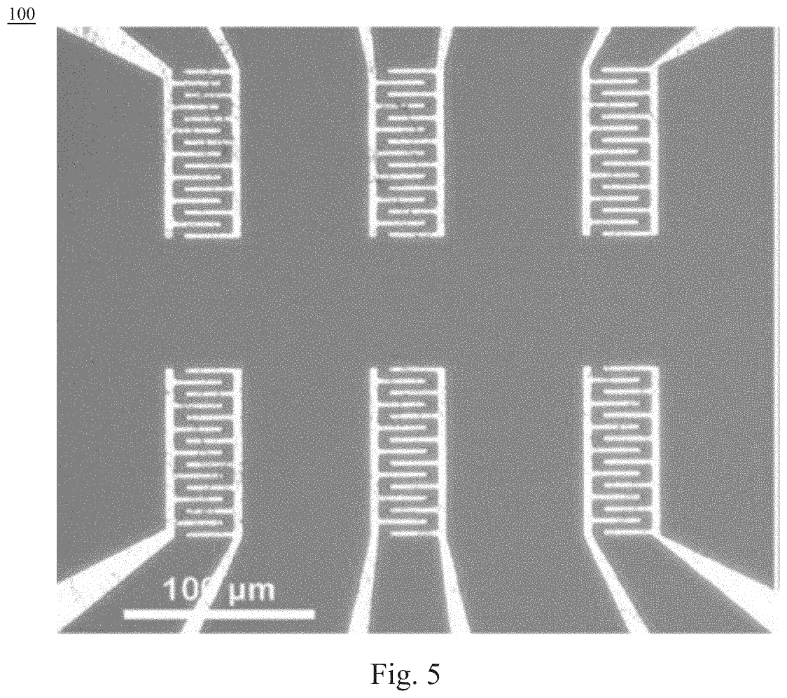

In some embodiments, in the fabrication of SiNW-FET devices, the CVD-grown SiNWs were deposited with a contact printing method in a lithography-predefined region on a photoresist (51805)-coated Si wafer of a 400 nm-thick oxide layer. Subsequently, the SiNWs-containing Si wafer was coated with photoresist layers of LOR5B and S1813. The metal contact regions were defined on the photoresists-coated Si wafer by a standard lithography procedure and cleaned with oxygen plasma (100 W, 50 sccm, 60 s). The silica sheath on SiNWs in the contact region (with electrodes) was removed with a buffered oxide etching solution. A thermal evaporation method was used to successively deposit the metal layers of Ni (70 nm in thickness) and then Al (100 nm in thickness) as the source and drain electrodes. The distance between the source and drain electrode is 3 μm. The SiNW-FET devices were further annealed in forming gas (10% H₂ and 90% N₂) at 360° C. for 3 min after removal of the photoresist to achieve good electrical contact between SiNWs and the metal electrodes. The aluminum oxide layer formed on the surface of the Al/Ni electrodes was used as an insulator to avoid electrical leakage during biosensing measurements. Shown in FIG. 5 is a top view of a single chip contains six as-fabricated MPC SiNW-FET devices. The leakage current of the MPC SiNW-FET device is from 10 pA to 100 pA measured when the MPC SiNW-FET device is covered by 1×PBS buffer solution at the solution gate-source voltage of 1 V.

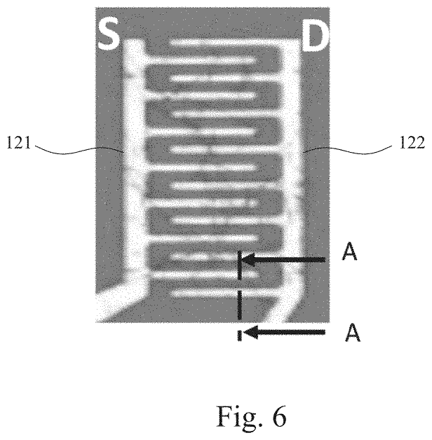

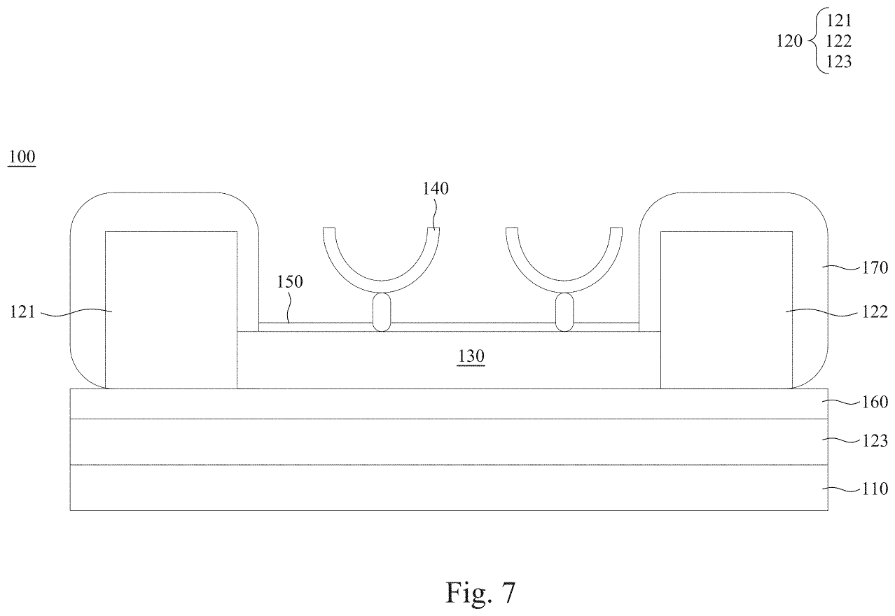

As shown in FIG. 5 to FIG. 7, FIG. 6 is a close-up view of FIG. 5 according to some embodiments of the present disclosure, and FIG. 7 is a cross-sectional view of the MPC SiNW-FET device according to some embodiments of the present disclosure. A nanoelectronic aptasensor 100 includes a substrate 110, a transistor 120, a plurality of silicon nanowires 130, a plurality of aptamers 140, a silane-based SAM 150, a dielectric layer 160, and an insulator 170.

The transistor 120 is disposed on the substrate 110, the transistor 120 includes a source electrode (S) 121, a drain electrode (D) 122, and a back gate electrode 123. In some examples, a thermal evaporation method was used to successively deposit the metal layers of Ni (70 nm in thickness) and then Al (100 nm in thickness) as the source and drain electrodes.

The plurality of silicon nanowires 130 are disposed on the dielectric layer 160, two ends of the plurality of silicon nanowires 130 respectively connect to the source electrode 121 and the drain electrode 122, and each one of the plurality of silicon nanowires 130 is separated to each other, in which each one of the plurality of silicon nanowires is single-crystalline.

The plurality of aptamers 140 is disposed on the plurality of silicon nanowires, and the aptamer includes a DNA sequence selected from the group consisting of derived sequences of SEQ ID NOs: 1, 2, 3, and 4, in which the derived sequences refer to that 3′ end and/or 5′ end of the derived sequences are modified, and the derived sequences have 90% identity to the SEQ ID NOs: 1, 2, 3, and 4, i.e., 91%, 92%, 93%, 94%, 95%, 96%, 97%, 98%, 99%, or any value between any two of these values.

The silane-based SAM 150 is disposed on the plurality of silicon nanowires 130, and the silane-based SAM 150 includes MPTMS, PTMS, or a combination thereof. In some examples, the plurality of aptamers 140 are conjugated with the MPTMS, and a ratio of the plurality of aptamers 140 and PTMS is 1:4. In some examples, the plurality of aptamers 140 are conjugated with the MPTMS by disulfide bonds. In some examples, the silane-based SAM 150 further includes PEG, and a ratio of the plurality of aptamers 140, PEG and PTMS is selected from 2:1:3, 1:1:3, 1:2:3, 1:4:3, or 1:6:3. In some examples, the plurality of aptamers 140 are conjugated with the MPTMS by disulfide bonds.

The dielectric layer 160 is disposed on the back gate electrode 123, and the back gate electrode 123 is disposed on the substrate 110. The source electrode 121 and the drain electrode 122 are respectively disposed on the dielectric layer 160,

The insulator 170 is formed on a surface of the source electrode 121 and a surface of the drain electrode 122 to avoid electrical leakage during biosensing measurements.

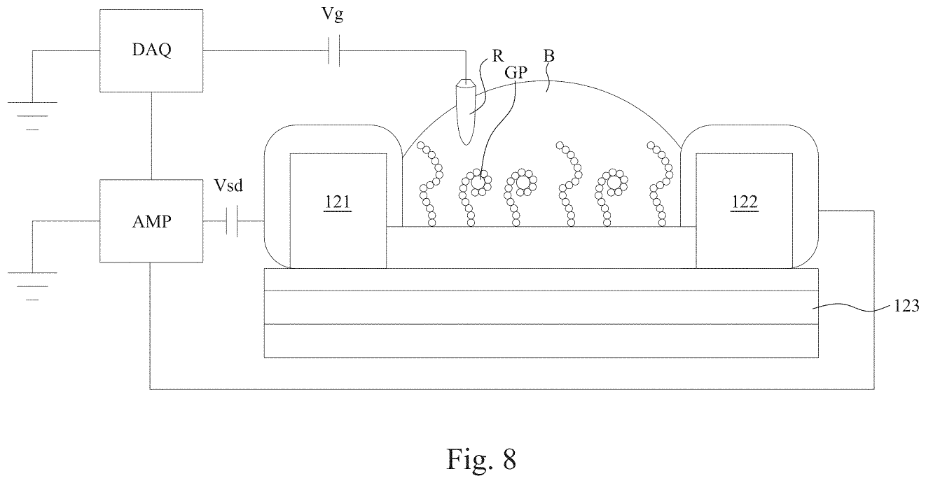

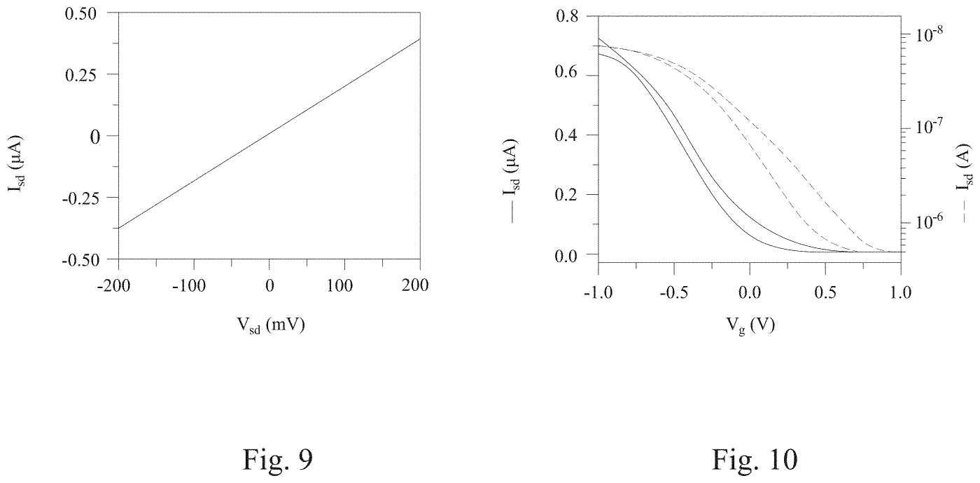

As shown in FIG. 8, a schematic illustration represents the experimental set-up for detecting GP or HbA1c by Apt_{GP}/SiNW-FET, where an Ag/AgCl reference electrode (R) in a buffer solution (B) was used as a solution-gate electrode. The electrical signals were acquired with a lock-in amplifier (AMP). The transfer curves (i.e., source-drain current vs. solution gate voltage (I_{sd}-V_{g}) plots) of the aptasensor were scanned at the bias voltage (V_{sd}) of 10 mV, and the solution-gate voltage (V_{g}) was controlled with a data acquisition (DAQ) system via the Ag/AgCl reference electrode (R). The output curve (i.e., a source-drain current vs. source-drain voltage (I_{sd}-V_{sd}) plot in FIG. 9) and transfer curve (i.e., a source-drain current vs. solution gate voltage (I_{sd}-V_{g}) plot in FIG. 10) of an MPC SiNW-FET were acquired with a lock-in amplifier (Stanford Research System, SR830) at V_{sd}=10 mV, a modulation frequency of 79 Hz, and a time constant of 100 ms. An Ag/AgCl electrode, immersed in the sample solution, was used as a solution-gate electrode with the voltage supplied by a data acquisition system (National Instruments, DAQ-NI2110). In biosensing measurements with MPC SiNW-FET devices, target analytes dissolved in a buffer solution were delivered to the SiNW-FET surface through a polydimethylsiloxane (PDMS) microfluidic channel driven by a syringe pump, or added directly onto the SiNW-FET devices surrounded by a PDMS wall. A representative output curve of an MPC SiNW-FET, recorded in ambient condition with a digital multimeter (Keithley 6487), shows an ohmic contact in FIG. 9. The transfer curve of an MPC SiNW-FET was measured in 1×PBS buffer solution with a lock-in amplifier (Stanford Research System, SR830) at V_{sd}=10 mV. The solution-gate voltage (V_{g}), scanned from −1 V to +1 V and back to −1 V, was applied via an Ag/AgCl reference electrode. The obtained transfer curve (I_{sd}-V_{g}) is respectively plotted on linear and logarithmic (dash line) scales in FIG. 10.

Surface Modification of SiNW-FETs

(A) An Apt_{GP}/SiNW-FET Aptasensor (without Co-Modifying PEG)

In some embodiments, prior to the modification of Apt_{GP} on an SiNW-FET device, the SiNW-FET surface was washed with ethanol and cleaned with oxygen plasma (25 W, 50 sccm, 60 s). Next, an ethanol solution (1% v/v) containing MPTMS and PTMS with a volume ratio of 1:4 was introduced onto the SiNW-FET device, where both MPTMS and PTMS were modified on the device surface (represented as MPTMS/SiNW-FET), followed by adding dithiothreitol (DTT) to reduce the possible disulfide linkages among the thiol groups of MPTMS. After 15 min of incubation, the device was rinsed with ethanol, dried under N₂, and heated at 100° C. for 15 min to ensure the formation of a silane-based SAM. Finally, the silane-based SAM-modified device was incubated for 1 hr in 1×PBS containing 1 μM Apt_{GP} and 16.7 mM DTT to form an Apt_{GP}/SiNW-FET aptasensor via a disulfide bond.

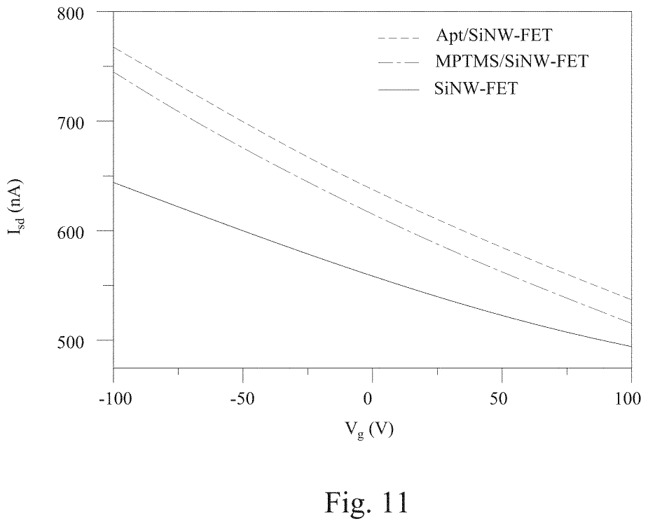

As shown in FIG. 11, the transfer curves were acquired for MPTMS/SiNW-FET (before modifying Apt_{GP}) (dash-dotted line) and Apt_{GP}/SiNW-FET (after modifying Apt_{GP}) (dash line) by scanning a back gate voltage. After modifying Apt_{GP}, the escalation of source-drain current (I_{sd}) was due to the gating effect of the negative charges on the phosphate backbone of Apt_{GP} to the p-type SiNW-FET. The transconductance (ΔI_{sd}/ΔV_{g}) is about 1100 nS.

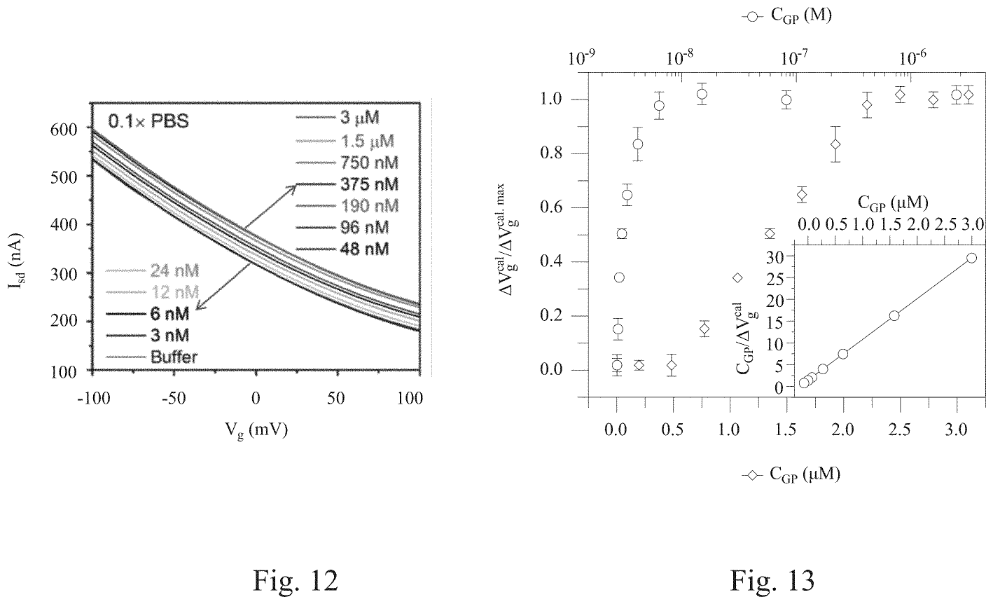

As shown in FIG. 12, we tested the detection capability of an Apt_{GP}/SiNW-FET aptasensor for GP by recording the shifts of transfer curve in response to various concentrations of GP (C_{GP}=3 nM to 3 μM) in 0.1×PBS (Debye length˜2.3 nm). To avoid the device-to-device variation in biosensing performance, the data obtained from FIG. 12 were converted to the calibrated response (ΔV_{g}^{cal}, at V_{g}=0 mV) as a function of C_{GP} and plotted in both linear and semi-log scales in FIG. 13. The inset of FIG. 13 shows the plot of C_{GP}/ΔV_{g}^{cal} against various C_{GP}, where Kd=46±7 nM of the GP-Apt_{GP} complex was determined by a least-squares fit of the measured data points to the Langmuir adsorption isotherm model. In FIG. 13, the limit of detection (LOD) of recognizing GP by an Apt_{GP}/SiNW-FET aptasensor is 6 nM, and the linear response regime in the semi-log plot (top abscissa) spans from 6×10⁻⁹ to 3.75×10⁻⁷ M.

(B) A PEG:Apt_{GP}/SiNW-FET Aptasensor (with Co-Modifying PEG)

In some embodiments, to enable an Apt_{GP}/SiNW-FET aptasensor to detect GP in high salt buffers, we co-modified Apt_{GP}, PEG-silane, and PTMS on an SiNW-FET (referred to as PEG:Apt_{GP}/SiNW-FET). Prior to the surface modification, an SiNW-FET was washed with ethanol and cleaned with oxygen plasma (25 W, 50 sccm, 60 s). The SiNW-FET device was then incubated for 15 min in an ethanol solution (1% v/v) containing an optimal ratio of MPTMS, PTMS, and PEG-silane, of which the ethanol solution was also mixed with 16.7 mM DTT to reduce the possible disulfide linkages among the thiol groups of MPTMS. Next, this silane-based SAM-modified device was rinsed with ethanol, dried under N₂, and heated at 100° C. for 15 min. Finally, the silane-based SAM-modified device was incubated in 1×PBS containing 1 μM Apt_{GP} and 16.7 mM DTT for 1 hr, where Apt_{GP} (with the 5′-SH) was immobilized to MPTMS via a disulfide bond to form a PEG:Apt_{GP}/SiNW-FET aptasensor.

To achieve the optimal detection sensitivity of a PEG:Apt_{GP}/SiNW-FET for recognizing GP in 1×PBS, we tested various ratios of Apt_{GP}:PEG:PTMS modified on the SiNW-FET surface, i.e., 2:1:3, 1:1:3, 1:2:3, 1:4:3, and 1:6:3. A ratio of Apt_{GP}:PEG:PTMS=1:2:3 could allow a PEG:Apt_{GP}/SiNW-FET to achieve the best LOD. Therefore, we prepared the PEG:Apt_{GP}/SiNW-FET devices with the ratio of Apt_{GP}:PEG:PTMS=1:2:3 for the subsequent experiments of detecting GP or HbA1c in high-salt buffer solutions.

### Example

Detection of HbA1c in Human Whole Blood Samples

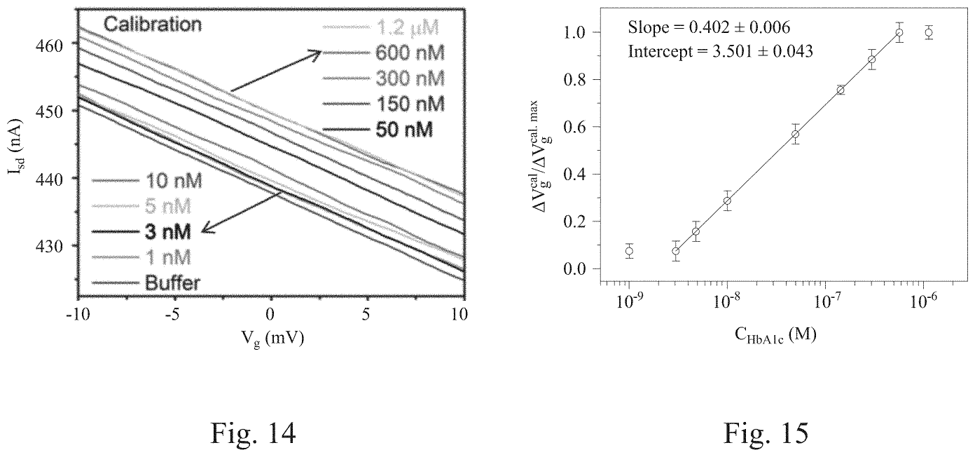

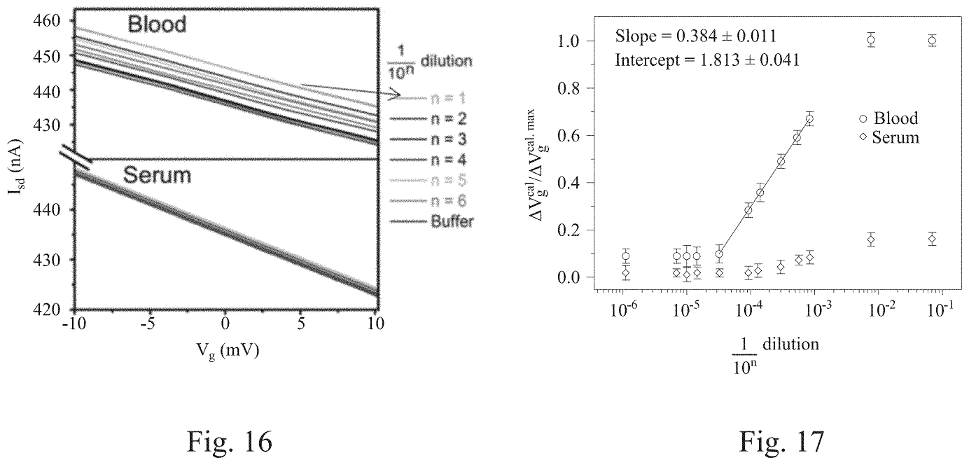

To demonstrate that a PEG:Apt_{GP}/SiNW-FET aptasensor could be used to detect HbA1c in clinical blood specimens, ten peripheral human blood samples (Subjects 1 to 10) were obtained and approved by the Institutional Review Board of Mackay Memorial Hospital, Taipei, Taiwan (19MMHIS289e) with written informed consent. We show a representative example of measuring HbA1c in the blood sample of Subject 1 by PEG:Apt_{GP}/SiNW-FET. Before detecting HbA1c in a lysed-blood sample, a PEG:Apt_{GP}/SiNW-FET aptasensor was calibrated by recording the transfer curves corresponding to various concentrations of HbA1c (C_{HbA1c}=0-1.2 μM in 1×PBS), as shown in FIG. 14. Similar to the conversion process from FIG. 12 to FIG. 13, the corresponding electrical response (ΔV_{g}^{cal}/ΔV_{g}^{cal,max}) as a function of C_{HbA1c} is obtained in FIG. 15. When measuring the HbA1c in blood samples with the calibrated PEG:Apt_{GP}/SiNW-FET aptasensor, a hemolytic blood sample was serially diluted 10¹ to 10⁶ times with 1×PBS to ensure that the CHbA1c levels lie within the linear response regime of the aptasensor without saturating the device. The recorded transfer curves of the diluted blood samples are displayed in the upper panel of FIG. 16 with the corresponding ΔV_{g}^{cal}/ΔV_{g}^{cal,max}-C_{HbA1c} plot shown in FIG. 17. By comparison, we also detected HbA1c in serum as a control test (with the transfer curves measured in the lower panel of FIG. 16 and the corresponding electrical responses presented in FIG. 17), where the serum was isolated from the tested blood sample by centrifugation. As shown in FIG. 17, the response of PEG:Apt_{GP}/SiNW-FET to serum is minor (only <10%) as compared to the diluted blood sample, reflecting that non-specific binding due to other components in blood is negligible.

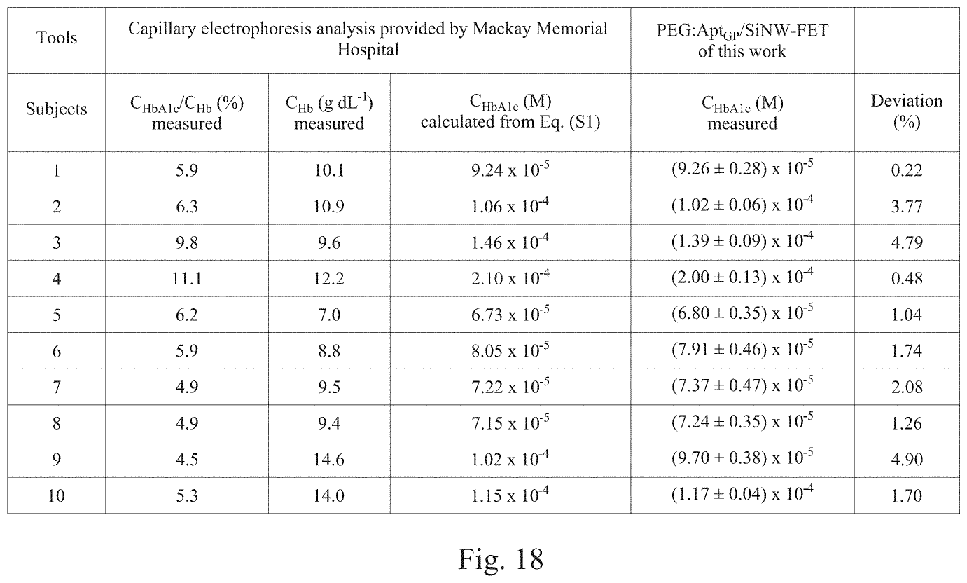

By taking the linear response regime of the 10³-10^{4.5}-fold dilution (i.e., a black line marked in FIG. 17 covering the range of 10⁻³-10^{-4.5} dilution) and calculating the measured C_{HbA1c} via the calibration plot (FIG. 15), we obtained C_{HbA1c}=(9.26±0.28)×10⁻⁵ M for Subject 1. This C_{HbA1c} value measured by PEG:Apt_{GP}/SiNW-FET is consistent with C_{HbA1c}=9.24×10⁻⁵ M examined by Mackay Memorial Hospital with a capillary electrophoresis method (Capillarys 3 Tera, Sebia), with a very small deviation of 0.22%. For the other nine blood samples of Subjects 2 to 10, we conducted HbA1c measurements by separate calibrated PEG:Apt_{GP}/SiNW-FET aptasensors. The C_{HbA1c} values of Subjects 1 to 10, determined by PEG:Apt_{GP}/SiNW-FET (in this laboratory) and measured with the conventional capillary electrophoresis method (in Mackay Memorial Hospital), are listed in FIG. 18 for comparison. The deviations for the C_{HbA1c} levels measured by these two tools are all within 5%, manifesting the accurate determination of C_{HbA1c} with the PEG:Apt_{GP}/SiNW-FET aptasensor developed in this study. Notably in the diagnosis of C_{HbA1c}, it generally takes much longer time for capillary electrophoresis than PEG:Apt_{GP}/SiNW-FET measurements.

While the disclosure has been described by way of example(s) and in terms of the preferred embodiment(s), it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures

What is claimed is:

1. A method of using an aptamer for detecting a glycated hemoglobin in a whole blood, comprising:
providing the aptamer comprising a DNA sequence selected from the group consisting of derived sequences of SEQ ID NOs: 1, 2, 3, and 4, wherein the derived sequences refer to that 3′ end and/or 5′ end of the derived sequences are modified, and the derived sequences have 90% identity to the SEQ ID NOs: 1, 2, 3, and 4;
contacting the aptamer and the whole blood; and
estimating a concentration of a conjugate of the aptamer and the glycated hemoglobin.

2. The method of claim 1, wherein the whole blood is from a human being.

3. The method of claim 1, wherein the glycated hemoglobin comprises a glycated peptide.

4. The method of claim 3, wherein the glycated peptide is D-fructose-valine-histidine-leucine-threonine-proline-glutamic acid.

5. The method of claim 4, wherein the D-fructose comprises β-D-fructopyranose, β-D-fructofuranose, α-D-fructofuranose, or α-D-fructopyranose.

6. A nanoelectronic aptasensor, comprising:
a substrate;
a transistor disposed on the substrate, the transistor comprising a source electrode, a drain electrode, and a gate electrode;
a plurality of silicon nanowires disposed on the substrate, two ends of the plurality of silicon nanowires respectively connecting to the source electrode and the drain electrode, and each one of the plurality of silicon nanowires separated to each other, wherein each one of the plurality of silicon nanowires is single-crystalline; and
a plurality of aptamers disposed on the plurality of silicon nanowires, and the aptamer comprising a DNA sequence selected from the group consisting of derived sequences of SEQ ID NOs: 1, 2, 3, and 4, wherein the derived sequences refer to that 3′ end and/or 5′ end of the derived sequences are modified, and the derived sequences have 90% identity to the SEQ ID NOs: 1, 2, 3, and 4,
wherein a leakage current of the nanoelectronic aptasensor is from 10 pA to 100 pA measured when the aptasensor is covered by one fold PBS buffer solution at a solution gate-source voltage of 1 V;
wherein a dissociation constant (Kd) without co-modification of polyethylene glycol (PEG) is from 39 nM to 53 nM.

7. The nanoelectronic aptasensor of claim 6, further comprising a silane-based self-assembled monolayer disposed on the plurality of silicon nanowires, and the silane-based self-assembled monolayer comprising 3-mercaptopropyl trimethoxysilane (MPTMS), propyltrimethoxysilane (PTMS), or a combination thereof,
wherein the plurality of aptamers are conjugated with the MPTMS, and a ratio of the plurality of aptamers and PTMS is 1:4.

8. The nanoelectronic aptasensor of claim 7, wherein the plurality of aptamers are conjugated with the MPTMS by disulfide bonds.

9. The nanoelectronic aptasensor of claim 7, wherein the silane-based self-assembled monolayer further comprising PEG,
wherein a ratio of the plurality of aptamers, PEG and PTMS is selected from the group consisting of 2:1:3, 1:1:3, 1:2:3, 1:4:3, and 1:6:3.

10. The nanoelectronic aptasensor of claim 7, wherein the plurality of aptamers are conjugated with the MPTMS by disulfide bonds.

11. The nanoelectronic aptasensor of claim 6, wherein each one of the plurality of silicon nanowires has a diameter in a range of 20 nm to 30 nm.

12. The nanoelectronic aptasensor of claim 6, wherein the plurality of silicon nanowires is p-type semiconductor or n-type semiconductor.

13. The nanoelectronic aptasensor of claim 6, wherein a detection range of a glycated hemoglobin in whole blood by the aptamers of SEQ ID NO: 1 is from 10⁻⁹ to 1.2×10⁻⁶ M.

14. The nanoelectronic aptasensor of claim 6, further comprising an insulator formed on a surface of the source electrode and a surface of the drain electrode.

15. The nanoelectronic aptasensor of claim 6, wherein a material of the source electrode and the drain electrode is selected from the group consisting of nickel and aluminum.
